(19) United States
(12) Patent Application Publication
(10) Pub. No.: US 2017/0340656 A1
(43) Pub. Date: Nov. 30, 2017

(54) Non-Invasive Imaging Methods for Patient Selection for Treatment with Nanoparticulate Therapeutic Agents

(71) Applicant: Merrimack Pharmaceuticals, Inc., Cambridge, MA (US)

(72) Inventors: Daryl C. Drummond, Lincoln, MA (US); Jonathan B. Fitzgerald, Arlington, MA (US); Ashish KaIra, Belmont, MA (US); Walid Kamoun, Arlington, MA (US); Stephan Klinz, Norwood, MA (US)

(21) Appl. No.: 15/622,329

(22) Filed: Jun. 14, 2017

Related U.S. Application Data
(63) Continuation of application No. 14/181,583, filed on Feb. 14, 2014, now abandoned.
(60) Provisional application No. 61/737,563, filed on Dec. 14, 2012.
(60) Provisional application No. 61/863,497, filed on Aug. 8, 2013.

(51) Int. Cl. A61K 31/704; A61K 31/4745; A61K 9/00; A61K 9/127

(52) U.S. Cl. CPC A61K 31/704; A61K 31/4745; A61K 9/0019; A61K 9/127

(57) ABSTRACT

Methods for providing treatment of pathologic conditions with nanoparticulate therapeutic agents are disclosed. Novel methods for determining liposomal deposition at sites of pathology using non-invasive imaging are also disclosed.

### CROSS-REFERENCE TO RELATED APPLICATIONS

This application is a continuation of U.S. patent application Ser. No. 14/181,583, filed on Feb. 14, 2014, which claims the benefit of and priority to U.S. Provisional Patent Application Nos. 61/737,563, filed Dec. 14, 2012 and 61/863,497, filed Aug. 8, 2013. Each of the foregoing applications are incorporated herein by reference in their entirety.

### BACKGROUND

Liposomal and other nanoparticulate therapeutic agents often exhibit long-circulating pharmacokinetics and will preferentially extravasate and accumulate in tissues perfused by hyper-permeable capillaries. Tumor blood vessels typically develop abnormally and have structural and physiologic defects leading to tumor capillary hyper-permeability. Capillaries at sites of pathologic inflammation (e.g., infection or inflammatory disease) may also exhibit hyper-permeability. As a result of the abnormal vasculature at sites of inflammation or tumor, the long-term presence of nanotherapeutics in the circulation may result in enhanced accumulation of nanoparticles such as liposomes at such sites of pathology. This preferential deposition of nanoparticulate therapeutic agents in tumors and sites of inflammation is referred to as the enhanced permeability and retention (EPR) effect. Different sites of pathology exhibit varying degrees of EPR.

Nanoparticle breakdown at the site of pathology releases the drug encapsulated in the nanoparticles locally, providing site-specific bioavailability of the active drug. In this setting nanoparticle breakdown is often an active process requiring cellular uptake. In some nanoparticulate agents, a pro-drug is entrapped that requires enzymatic activation for optimal function, e.g., hydrolysis by esterases such as those localized preferentially in intracellular organelles such as endosomes and lysosomes. In tumors and at other sites of inflammation, liposomes are often actively taken up (e.g., via phagocytosis) by white blood cells such as monocytes and macrophages, where prodrug activation may take place.

Liposomes or lipidic nanocarriers can be designed to exhibit a wide range of physicochemical properties that in turn impact their pharmacologic properties, including circulation lifetimes and degree of tumor deposition. The majority of liposomes developed to date display either a fairly rapid clearance from the circulation or rapid release of their encapsulated payload while in circulation.

A recently developed class of liposomes, that represents a minority of liposomal drugs in clinical development, exhibit a combination of long circulation lifetimes (t_{1/2}>6-10 h in mice, >12-24 h in humans) and highly stable retention of encapsulated drug while in the circulation (T_{1/2} of release >18-24h). These included a pegylated liposomal doxorubicin (PLD) prepared from mPEG-DSPE, hydrogenated soyphosphatidylcholine (HSPC) and cholesterol and stabilized with intraliposomal ammonium sulfate, and similar formulations decorated on their surface with antibody-fragments specific for ErbB2 or EGFR cell surface receptors (Park et al., Clin Cancer Res. 2002; 8(4):1172-1181; Drummond et al., Pharmacol Rev. 1999; Dec; 51(4):691-743; Mamot et al., Cancer Res. 2005; 65(24):11631-11638). These also include liposomal formulations of irinotecan, topotecan, vincristine, and vinorelbine that have been stabilized through association (e.g., gelation) with an (optionally high density) sulfated sugar or polyol, e.g., with sucroseoctasulfate to form the sucrosofate salt, in the liposome interior (see U.S. Pat. Nos. 8,147,867 and 8,329,213). Other members of this class of liposomes are nucleic acid formulations stabilized with mPEG-DSPE and a unique solvent condensation protocol (Hayes et al., 2006; Biochim Biophys Acta. 2006 Apr; 1758(4): 429-442, also see U.S. Pat. No. 8,496,961 and U.S. application Ser. No. 13/967,664, filed 15-Aug-2013). This class of liposomes or lipidic nanocarriers will benefit from prediction of liposome deposition at sites of inflammation, infection, and/or high macrophage concentration.

An exemplary liposomal anti-neoplastic agent, MM-398, also known as PEP02 and as irinotecan (sucrosofate) liposome injection, comprises the pro-drug irinotecan encapsulated in a liposomal drug delivery system. MM-398 is prepared as described, e.g., in U.S. Pat. No. 8, 147,867. This stable nanoliposomal formulation of irinotecan has long-circulating pharmacokinetics. MM-398 liposomes accumulate in tumor-associated macrophages (TAMs). Once internalized by TAMs, irinotecan is believed to be released from within the liposomes and activated by cellular carboxyl esterase activity to yield SN-38, which is generally 100-1000× more active as an inhibitor of cancer cell proliferation than is irinotecan. In this fashion, TAMS are believed to play an essential role in establishing high concentrations of SN-38 in an MM-398 treated tumor, an outcome designed to improve clinical outcomes in treated cancer patients. Thus, tumor deposition and accumulation, liposome breakdown, and conversion of irinotecan into SN-38 are all believed to be critical and rate-limiting elements of MM-398 trafficking and disease modification.

Other exemplary liposomal anti-neoplastic agents include doxorubicin liposome injection (DOXIL or CAELYX) and MM-302-HER2-targeted immunoliposomal doxorubicin. Immunoliposomes are antibody (typically antibody fragment) targeted liposomes that can provide advantages over non-immunoliposomal preparations because they are selectively internalized by cells bearing cell surface antigens targeted by the antibody. Details of antibodies and immunoliposomes are described, for example, in the following US patents: U.S. Pat. Nos.7,871,620, 6,214,388, 7,135,177, and 7,507,407 (“Immunoliposomes that optimize internalization into target cells”); U.S. Pat. No. 6,210,707 (“Methods of forming protein-linked lipidic microparticles and compositions thereof”); U.S. Pat. No. 7,022,336 (“Methods for attaching protein to lipidic microparticles with high efficiency”) and U.S. Pat. No. 7,892,554 and 7,244,826 (“Internalizing ErbB2 antibodies.”).

Ferumoxytol (Feraheme®, AMAG Pharmaceuticals, Inc., referred to herein as “FMX”) is a preparation of polyglucose sorbitol carboxymethylether coated magnetite (superparamagnetic iron oxide) nanoparticles (17-31 nm average diameter). Marketed for the treatment of iron deficiency anemia in patients with chronic renal failure, FMX is administered to adult patients at a dosage of 510 mg by intravenous injection followed by a second 510 mg intravenous injection 3 to 8 days later. FMX exhibits a prolonged residence time in the bloodstream (i.e., within the intravascular space). When performing MRI studies in subjects given this agent for the treatment of their anemia, exquisite angiographic images can be obtained up to 24 hours post injection. Contrast enhancement of the intravascular space following FMX administration has provided a novel approach to imaging pathologic conditions that involve the vascular tree such as stroke, vascular malformations, and chronic renal disease. In addition, this agent has been used in patients with CNS malignancies to measure tumor vascularity and the response to chemotherapy. Currently, the FDA has designated this agent as an orphan drug for brain tumor imaging.

FMX is significantly smaller than most liposomal therapies, which have an average size of 70-200 (more commonly 80-120) nm. Size dependence for clearance rates of liposomes from the blood stream and with respect to tumor deposition has been well characterized. Generally liposomes smaller than 50 nm are cleared significantly more slowly than liposomes with a size of 100 nm or greater. In addition, many smaller nanoparticles accumulate less efficiently in tissues due to an apparently high efflux rate from tissues of smaller particles.

Because liposomes and other nanoparticulate therapeutic agents can vary in size, and nanoparticle size may impact EPR levels, it would be desirable to be able to predict EPR-mediated accumulation of nanoparticles of particular sizes at sites of pathology in patients. Furthermore, some imaging agents may saturate the reticuloendothelial system, resulting in a significant decrease in the clearance rate of subsequently administered nanotherapeutics. This may dramatically increase the systemic exposure of the nanotherapeutics, creating a significant safety risk to the patient. Waiting for treatment until the effect on clearance has diminished is impractical for cancer patients, whose fast growing cancers require rapid implementation of new treatment options.

There is thus a need for safe and effective methods of screening patients to identify those patients with sites of pathology that are predicted to exhibit nanoparticle accumulation or not to do so, which methods do not themselves alter nanoparticle accumulation. Patients identified as having sites of pathology that are predicted to exhibit nanoparticle accumulation would be considered more likely to respond to nanoparticulate therapeutic agents. Patients identified as having sites of pathology that are predicted not to exhibit nanoparticle accumulation would be considered less likely to respond to nanoparticulate therapeutic agents. Treating patients in accordance with such identifications would avoid the administration of sub-optimal therapeutic treatments to patients in need of therapy. The following disclosure addresses these needs and provides additional benefits.

### SUMMARY OF THE INVENTION

The present invention provides methods for selecting and providing liposomal therapy for pathologic conditions based on novel methods for determining liposomal deposition at sites of pathology using non-invasive imaging. Such methods are useful in the selection of treatment for various conditions including localized infectious, inflammatory and proliferative diseases.

In certain aspects, a method is provided for selecting and providing pharmaceutical treatment to a patient for a localized infectious, inflammatory, or neoplastic condition, the method comprising identifying one or more locations of infection, inflammation or neoplasia in a patient, and subsequently, obtaining at least one contrast-enhanced MRI image of a first location of the one or more locations, and subsequently, selecting an anti-infective, anti-inflammatory, or anti-neoplastic pharmaceutical agent and treating the patient with the selected pharmaceutical agent, wherein, the contrast-enhanced MRI image is obtained following administration of an MRI contrast agent comprising an MRI contrast-enhancing amount of superparamagnetic iron oxide nanoparticles of about 15-30 nm in diameter, and if the contrast-enhanced MRI image shows contrast enhancement within at least one of the one or more locations, the selected pharmaceutical agent is a liposomal therapeutic agent that is formulated as nanoparticles or nanoliposomes of about 70 to about 200 nm in diameter, but if the contrast-enhanced MRI image does not show contrast enhancement at first location, the selected pharmaceutical agent is not formulated as nanoparticles or nanoliposomes of about 70 to about 200 nm in diameter, or, in another embodiment, the selected pharmaceutical agent is not formulated as nanoparticles or nanoliposomes of any size. In certain embodiments, the method comprises diagnosing a localized infectious, inflammatory, or neoplastic condition in the patient.

In certain embodiments, the MRI contrast agent is ferumoxytol. In certain embodiments, the ferumoxytol is administered at 5 mg iron per kg. In certain embodiments, the patient is an adult and 100-1000 milligrams (iron) of ferumoxytol are administered to the patient. In certain embodiments, the patient is an adult and 510 milligrams of ferumoxytol are administered to the patient. In certain embodiments, the contrast-enhanced MRI image is obtained no more than 72, 96, 120, 144 or 168 hours after administration of the superparamagnetic iron oxide nanoparticles. In certain embodiments, the contrast-enhanced MRI image is obtained from 72-96, 96-120, 120-144, or 144 to 168 hours after administration of the superparamagnetic iron oxide nanoparticles.

In other aspects, a method is provided for selecting and providing pharmaceutical treatment to a patient for a localized infectious, inflammatory, or neoplastic condition, the method comprising identifying two or more locations of infection, inflammation or neoplasia in the patient, and subsequently, obtaining a first contrast-enhanced MRI image of a first location of the one or more locations, obtaining a second contrast-enhanced MRI image of a second location of the two or more locations, and subsequently, selecting an anti-infective, anti-inflammatory, or anti-neoplastic pharmaceutical agent and treating the patient with the selected pharmaceutical agent; wherein, the first and second contrast-enhanced MRI images are obtained following administration of an MRI contrast agent comprising an MRI contrast-enhancing amount of superparamagnetic iron oxide nanoparticles of about 15-30 nm in diameter, and if both the first and the second contrast-enhanced MRI images show contrast enhancement within both the first location and second location, the selected pharmaceutical agent is an agent that is formulated as nanoparticles or nanoliposomes of about 70 to about 200 nm in diameter (a liposomal therapeutic agent), but if both the first and the second contrast-enhanced MRI images do not show contrast enhancement within both the first location and second location, the selected pharmaceutical agent is an agent that is not formulated as nanoparticles or nanoliposomes of about 70 to about 200 nm in diameter.

In one embodiment of either of the above aspects, the method comprises identifying at least one region of interest in a patient (e.g., a region having a localized infection, inflammation, or neoplasm) and obtaining a contrast-enhanced MRI image of a region of interest predictive of the deposition of a liposomal anti-infective, anti-inflammatory, or antineoplastic therapeutic agent in the region of interest in the patient, e.g., a region of localized infection, inflammation, or a solid tumor in a cancer patient, and then for determining whether or not such a liposomal anti-infective, anti-inflammatory, or antineoplastic liposomal therapeutic agent will accumulate in the region of localized infection, inflammation, or solid tumor in the patient, and subsequently, selecting an anti-infective, anti-inflammatory, or anti-neoplastic liposomal therapeutic agent and treating the patient with the selected pharmaceutical agent. These methods comprise obtaining an MRI image of the tumor in the patient where, between 24 hours and 72 hours prior to MRI imaging, an MRI contrast agent consisting of an ultra-small superparamagnetic iron oxide particle (USIOP) preparation comprising particles with an average size of about 15 to about 30 nm in diameter is administered, e.g., via a parenteral route, to the patient in an amount sufficient to provide MRI contrast and the MRI imaging is performed no more than 168 hours after administration of the contrast agent whereby the MRI image obtained is contrast-enhanced. In certain aspects, if the MRI image of the site of pathology or region of interest shows areas of enhanced contrast (e.g., as compared to images obtained of normal muscle, of other tissues where the contrast agent does not accumulate, or as compared to images obtained without pretreatment with an MRI contrast agent), the patient is identified as being suitable for treatment with the liposomal therapeutic agent. In certain aspects the patient so identified is so treated.

In some embodiments of either of the aspects above, the liposomal anti-infective, anti-inflammatory, or antineoplastic pharmaceutical agent comprises liposomes with an average diameter of, e.g., 70-200, or 75-160, or 80-200 nm, or e.g., about 80 nm to about 120 nm (e.g., an average size of about 100 nm in diameter with an average range of about 80 nm to about 120 nm). In one embodiment, the liposomal therapeutic comprises irinotecan or doxorubicin. In another embodiment, the liposomal therapeutic comprises a targeting antibody. In one embodiment, the liposomal therapeutic agent is MM-398 (irinotecan sucrosofate liposome injection). In another embodiment, the liposomal therapeutic agent is MM-302 (Her2-targeted liposomal doxorubicin). In one embodiment, the first contrast-enhanced MRI image shows contrast enhancement within the first location, the neoplastic condition comprises at least one tumor and the pharmaceutical agent is a liposomal anti-neoplastic agent. In another embodiment, the tumor is a non-small cell lung cancer (NSCLC) tumor, a triple negative breast cancer (TNBC) tumor, a colorectal cancer (CRC) tumor, an ER/PR positive breast cancer tumor, a pancreatic cancer tumor, an ovarian cancer tumor, a small cell lung cancer tumor, a gastric cancer tumor, a GEJ adenocarcinoma, a head and neck cancer tumor, a cervical cancer tumor, or Ewing's sarcoma and the selected pharmaceutical agent is a liposomal formulation of irinotecan.

In one embodiment of either of the aspects above, the contrast-enhanced MRI image is obtained no more than 72, 96, 120, 144 or 168 hours after administration of the superparamagnetic iron oxide nanoparticles. In another embodiment, the contrast-enhanced MRI image is obtained from 72-96, 96-120, 120-144, or 144 to 168 hours after administration of the superparamagnetic iron oxide nanoparticles.

In one embodiment of either of the aspects above, the MRI image is obtained using a magnetic field strength of 1-10 Tesla, e.g., 1.5 Tesla or 3 Tesla. An exemplary USIOP preparation is FMX (ferumoxytol). In some embodiments, the MRI contrast agent is ferumoxytol. In one embodiment, the ferumoxytol is administered at 5 mg/kg. In another embodiment, the patient is an adult and 100-1000 milligrams of ferumoxytol are administered to the patient. In yet another embodiment, the patient is an adult and 510 milligrams of ferumoxytol are administered to the patient.

In certain embodiments of either of the aspects above, the liposomal therapeutic agent is an anti-neoplastic agent. An exemplary anti-neoplastic agent is liposomal irinotecan, e.g., irinotecan sucrosofate liposome injection. Another exemplary liposomal anti-neoplastic agent is liposomal doxorubicin, e.g., doxorubicin lipid complex injection or antibody-targeted doxorubicin liposomes (e.g., with anti-HER2 antibody attached to liposome surface).

In one embodiment, the tumor exhibits (e.g., in biopsy sections) an elevated level of tumor associated macrophages (TAMs). Tumors with 3 or more, or 4 or more, or 5 or more macrophage-marker (e.g., CD68) positive cells per high power field (hpf) are considered to exhibit elevated levels of TAMs. In some embodiments the elevated level of TAMs corresponds to 3 or more, or 4 or more, or 5 or more cells that are both CD68 positive and PCNA (proliferating cell nuclear antigen) positive per hpf. Macrophage marker and (optionally) PCNA positive TAMs in tumor sections may be identified and numbers per hpf determined as described, e.g., per Mukhtar, et al., Cancer Res. Breast Cancer Research and Treatment; 2011;130(2):635-644. The tumor may be any tumor, e.g., a tumor of a non-small cell lung cancer (NSCLC), a triple negative breast cancer (TNBC), a colorectal cancer (CRC), an ER/PR (estrogen receptor and/or progesterone receptor) positive breast cancer, a pancreatic cancer, an ovarian cancer, a small cell lung cancer, a gastric cancer, a GEJ (gastro-esophageal junction) adenocarcinoma, a head and neck cancer, a cervical cancer, or a Ewing's sarcoma.

In various embodiments the imaging is performed (about) 24, 48, or 72 hours after the administration of the contrast agent.

Other features and advantages will be apparent from the following disclosure and the claims.

### BRIEF DESCRIPTION OF THE DRAWINGS

FIG. 1A. Provides areas of ferumoxytol signal in areas of low liposome deposition in two primary models of pancreatic tumors.

FIG. 1B. Provides areas of ferumoxytol signal in areas of high liposome deposition in two primary models of pancreatic tumors.

FIG. 1C. Image providing the spatial distribution of DiI5 liposomes.

FIG. 1D. Image providing the spatial distribution of FMX.

FIG. 1E. Image providing the spatial distribution of F4/80.

FIG. 2A. A graph showing the overall DiI5 liposome uptake expressed as the percent of the total population of cells that are DiI5 positive, after injection of HT-29 tumor-bearing mice with varying amounts (0, 20, or 50 mg/kg) FMX followed by DiI5 liposomes, indicating that FMX pre-treatment has no effect.

FIG. 2B. A graph showing that the mean fluorescence intensity (MFI) of DiI5 liposomes was higher in CD11b+myeloid cells as compared to MFI in EpCAM+tumor cells, after injection of HT-29 tumor-bearing mice with varying amounts (0, 20, or 50 mg/kg) FMX followed by DiI5 liposomes, and indicating that FMX pre-treatment has no effect.

FIG. 3. As described in Example 3, below, FIG. 3 indicates the degree of accumulation of SN-38 in tumors after MM-398 administration following either no pretreatment, or pretreatment with 20 or 50 mg/kg of FMX. No effect of FMX pre-treatment was observed.

FIG. 4 (see Example 4, below) shows the pharmacokinetics of MM-302 either without pre-dose, or following 72 hours pre-dose with iodine-loaded liposomes at 3, 10, and 30 times the lipid dose of the F5-ILs-Dox. “% i.d.” indicates the percentage of the initial dose remaining. A large effect of iodine-loaded liposome pretreatment was observed.

FIG. 5 (see Example 5 below) shows the uptake of FMX 24 hr after FMX (20 mg/kg) intravenous injection by human HT29, A549 and A2780 tumors grown subcutaneously in SCID mice as measured by MRI and T2*analysis. The FMX levels are plotted on the x-axis relative to the irinotecan (CPT-11) concentration in ng/g (as displayed on the y-axis) measured by HPLC in total tumor lysates from the imaged tumors harvested 72 hrs after the injection of MM-398 (10 mg/kg) into the same mice.

FIG. 6A shows the FMX pharmacokinetics in patient plasma after injection of 5 mg/kg FMX.

FIG. 6B shows the FMX signals in both plasma and four different tumor lesions of a single patient.

FIG. 6C shows the output of a pharmacokinetic model that can describe the measured FMX plasma and tumor lesion data through two parameters, a tissue permeability parameter and a tissue binding parameter that together describe lesion-specific FMX levels.

FIG. 7 (see Example 8 below) describes IHC results from tissue sections derived from patient biopsies after treatment with FMX. Macrophages are stained with an anti-CD68 antibody (clone PG-M1, DAKO), while iron detection in an adjacent tissue section is by Prussian Blue staining.

FIG. 8 (see Example 9 below) shows the relationship between the FMX signal in select regions-of-interest of tumor lesions measured at 24 hr and the corresponding irinotecan (CPT-11) levels (ng/g) measured in biopsies from the vicinity of those regions.

FIG. 9 is an image of an MRI standard curve or “phantom”, which shows a T2* weighted MRI with TE of 13.2 ms with phantom tubes from 0-200 μg/ml ferumoxytol.

### DETAILED DESCRIPTION

Provided herein are methods for obtaining images indicating whether or not a liposomal therapeutic agent will accumulate at a site of malignancy or inflammation, as well as methods of patient selection and treatment informed by such images.

A method is provided in which nanoparticulate MRI contrast agents are used to obtain contrast-enhanced MRI images of a site of pathology in a patient and the images are used to predict whether the site of pathology in the patient will have low or high deposition of liposomal drugs, with the prediction allowing a determination of whether or not a liposomal drug should be administered to the patient.

Because FMX particles are much smaller than therapeutic liposomes, it was surprising to find that FMX particle deposition in pathologic tissues is predictive of the deposition in such tissues of a much larger therapeutic agent (e.g., a liposomal agent). It is demonstrated herein that tissues that display FMX contrast enhancement upon MRI are more likely to accumulate deposition of liposomal therapeutics (comprising liposomes averaging, e.g., 70-200, or 75-160, or 80-200 nm in diameter) than those that do not exhibit FMX MRI contrast enhancement.

As FMX has been demonstrated to be safe for intravenous administration to patients and is shown herein not to interfere with nanoliposome therapies if used as an imaging agent, even within 1-4 hours prior to administration of nanoliposomal therapeutics, these results indicate that FMX MRI allows for selection patients who will (or will not) benefit from nanoliposomal therapy.

Contrast enhancement is detected, typically by a radiologist, in a patient undergoing imaging. Contrast enhancement is detected when a region of interest (such as a region comprising or within a tumor or site of infection or inflammation) shows greater contrast than one or more regions not expected to show enhanced contrast (such as a tumor-free region in a tissue that does not typically comprise high levels of macrophages, such as liver and spleen do). For example, if a region of interest in such an imaging patient shows an observable enhancement over a tumor, infection and inflammation-free region in a tissue that is not expected to show enhanced contrast, the patient is identified as a candidate for treatment with a liposomal therapeutic. In some embodiments, contrast enhancement is detected for a region of interest as compared to the contrast enhancement observed in a similar region in at least one other patient, e.g., a comparator patient (i.e., a patient with a similar, per type and location, tumor, infection or inflammation) or a population of comparator patients, which patient or patients have also undergone the ferumoxytol imaging procedure. For example, for a given time point (e.g., 20, 24, 30, 36, 48, 72, 96, 120, 144, or 168 hours) after an imaging patient is injected with ferumoxytol, if the MRI contrast enhancement of the region of interest in the imaging patient is greater than the mean (or median) contrast enhancement observed for one or more regions of interest in the comparator patient or the comparator patient population, then the imaging patient is identified as a candidate for treatment with a liposomal therapeutic. In another embodiment, in a patient having more than one region of interest, an image is taken of a plurality of, or of all, regions of interest. If the plurality or all regions of interest show contrast enhancement, then the patient is identified as a candidate for treatment with a liposomal therapeutic. In certain embodiments, the patient so identified as a candidate is treated with an effective amount of a liposomal therapeutic.

The liposomal therapeutic agent to be administered in accordance with the methods disclosed herein retains the encapsulated drug in the bloodstream with a T1/2 of release of at least 18 hours, 24 hours, or 48 hours following injection. The average particle size (diameter) of the nanotherapeutic to be delivered is between 70-200 nm, e.g., 80-120 nm or 75-200 nm.

For an imaging agent to be used to be safely used to predict the deposition of a subsequently delivered nanotherapeutic, such as a nanoliposome, the imaging agent must not significantly alter the pharmacokinetics or pharmacodynamics of the nanotherapeutic with which the patient is to be treated.

### EXAMPLE 1

### FMX and Nanoliposomes Deposit Within the Same Areas of Tumors

In order to analyze the microdistribution of FMX as it compares to distribution of liposomes, two primary human pancreatic cancer tumor samples were passaged as flank xenografts through nu/nu mice. A tumor thus passaged 10 time produced xenograft tumor model 254, while another passaged 6 times produced xenograft tumor model 269. Tumor fragments thus prepared were implanted in the experimental mice (n=4 for model 254 and n=6 for model 269) and allowed to grow to ˜200-300 mm³.

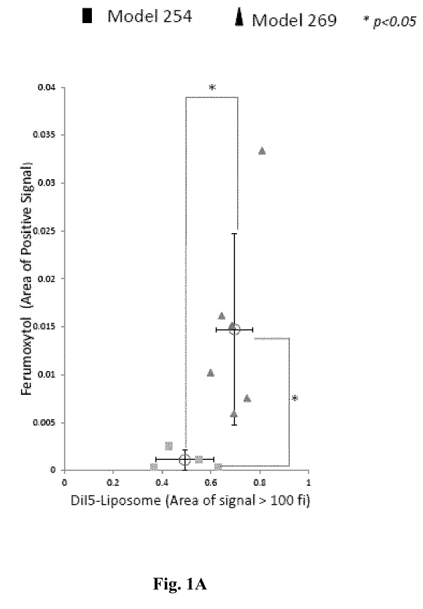

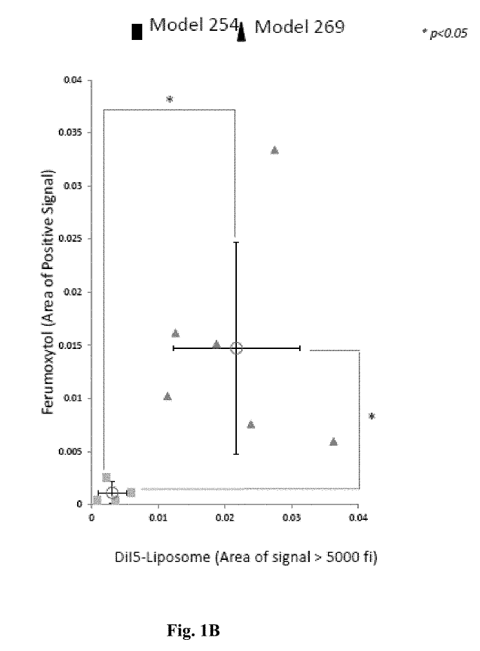

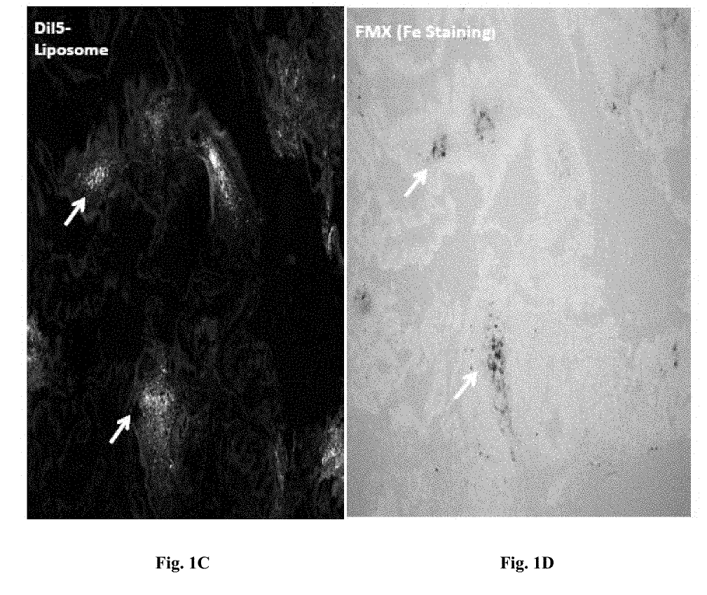

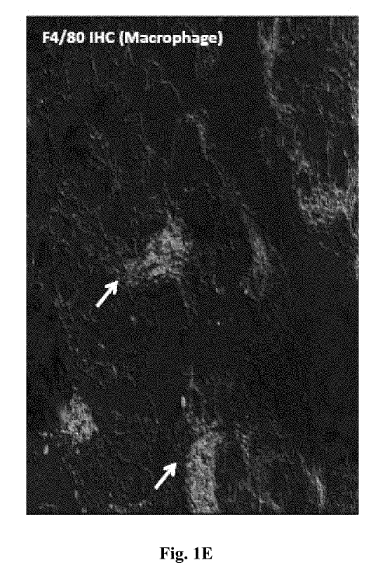

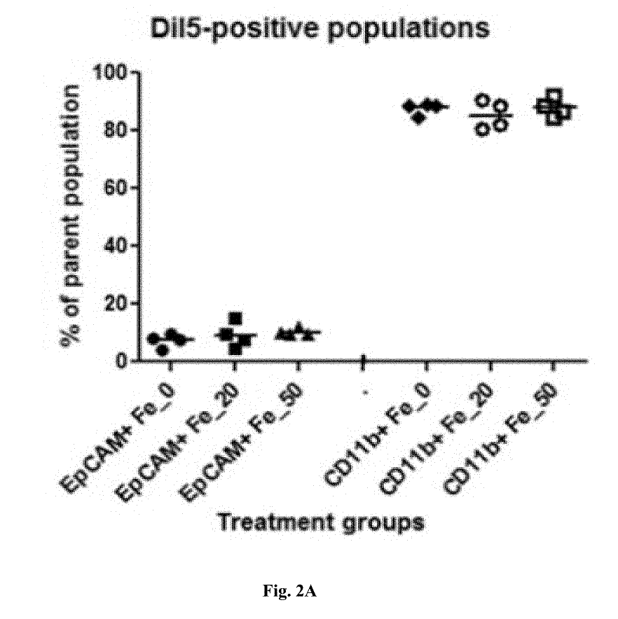

Tumor-bearing mice thus prepared were injected with FMX at 25 mg/kg followed by an injection of DiI5-labeled liposomes 24 hours later at 40 micromoles of phospholipid per kg of body weight. Mice were sacrificed at 48 hours post-FMX injection (24 hours post DiI5-liposome injection) and tumor sections were prepared and assessed by Prussian Blue staining (Aperio® ScanScope AT® whole slide scanner) for FMX and by fluorescence microscopy (Aperio® ScanScope FL® whole slide scanner) for DiI5 liposomes. Each resulting scan measured fluorescence intensity (fi) and was segmented and fraction of area that was FMX or DiI5 positive was calculated using MatLab®. The fraction of liposome-positive area was calculated from the images using simple thresholding (fi>5000 for areas of high liposome deposition and fi>100 for. areas of low liposomal deposition). Model 269 showed statistically significantly higher levels of tumoral deposition of the liposomes when compared to animals in model 254. Deposition of FMX followed the same trend and correlated well with the deposition of the liposomes (FIG. 1A). In order to analyze the spatial distribution of the liposome and FMX, corresponding areas were identified in serial sections of the same mouse stained for FMX, imaged for liposomes, or immunohistochemically stained for F4/80, a murine macrophage marker. Liposomal spatial distribution showed microdistribution with hot spots (areas of high liposomal deposition) scattered throughout the tumor. FMX showed a similar pattern and co-localized with the hot spots of liposomes. Macrophages were present at relatively high levels within these tumors and spatially correlated with the liposomes and the FMX (FIG. 1B). Microscopy confirmed the co-localization of liposomes (FIG. 1C), FMX (FIG. 1D) and F4/80 IHC-detected myeloid cells (e.g., macrophages-FIG. 1E).

### EXAMPLE 2

### Effects of FMX on Phagocytosis by Macrophages

HT-29 tumor-bearing mice were injected with FMX followed by injection of liposomes pre-labeled with DiI5 (a fluorescent dye). HT-29 xenografts were developed by inoculating 10 million HT-29 colorectal adenocarcinoma cells (ATCC) per mouse in SCID mice. Once tumors were well established (˜200-300mm³) treatment was initiated. Mice were administered a single dose of ferumoxytol (20 mg/kg or 50 mg/kg) followed by an i.v. dose of MM-398 equivalent to 20 mg irinotecan HCL/kg, or DiIC18(5)-DS (DiI5) labeled liposomes at 40 μmol phospholipid/kg, for HPLC and FACS analysis respectively. HT-29 tumors were collected at end of the study for IHC and HPLC analysis. Flow cytometry was performed on a BD FACSCalibur® instrument. Analysis of irinotecan levels in tumor tissues was as described by Noble, et al, Cancer Res. 2006;66:2801-2806. Water was added to tissues at a 20% (w/v) ratio, and tissues then homogenized with a mechanical homogenizer in an ice bath. Homogenates were extracted for the lactone form of irinotecan with an acidic methanol solution by vortexing and centrifugation at 13,000 rpm for 10 minutes, with the supernatants then transferred to autosampler vials for Dionex™ (Thermo Scientific) high-pressure liquid chromatography (HPLC) analysis.

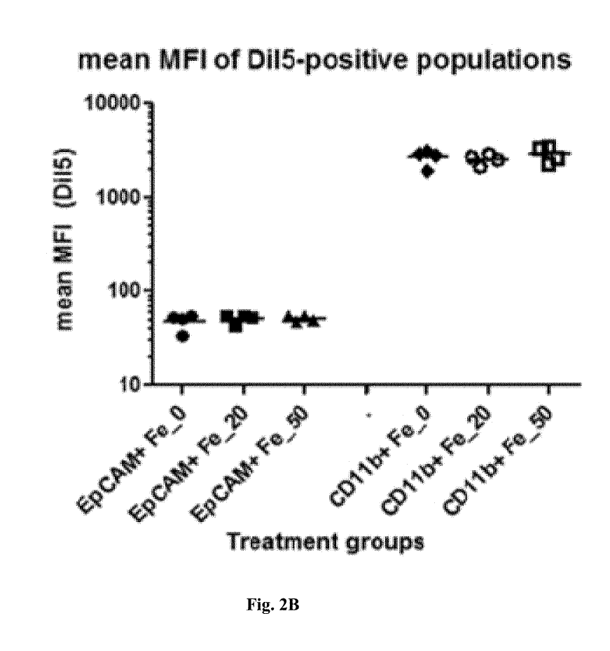

FACS analysis indicated that there was no effect of FMX (Fe) on overall DiI5 liposome uptake (FIG. 2A) in either EpCAM+ (tumor) cells or CD11b+ (myeloid) cells (e.g., macrophages). The mean fluorescence intensity (MFI) of DiI5 liposomes (FIG. 2B) was higher in CD11b+ myeloid cells and remained unaltered by FMX as compared to MFI in EpCAM+ tumor cells.

### EXAMPLE 3

### Effects of FMX on Pharmacology of Subsequently Administered Liposomal Irinotecan

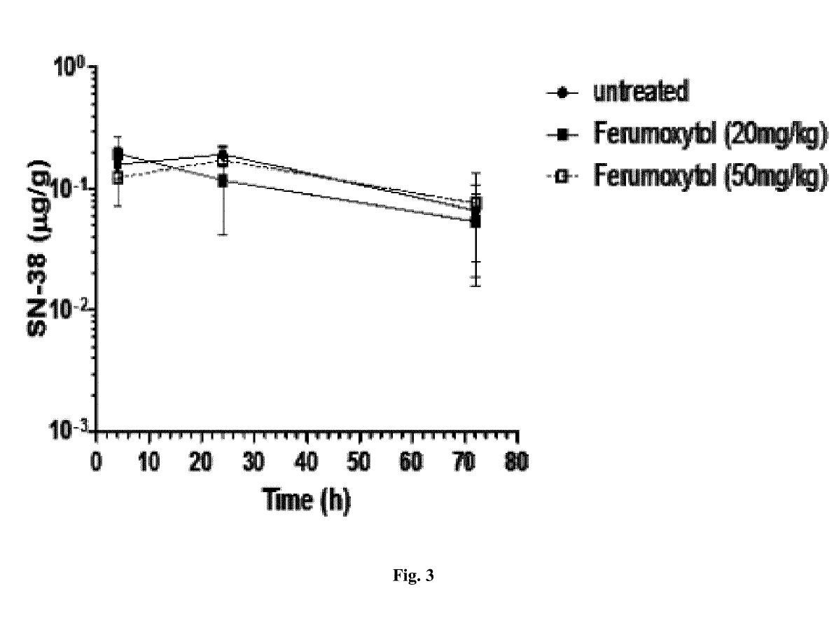

HT-29 tumor-bearing mice were injected with imaging agent (FMX) followed by injection of therapeutic liposomes as described in the preceding Example. The liposomes were MM-398 liposomes and were administered at 20 mg/kg and at 50 mg/kg with tumor samples being taken at 2, 24 and 72 hours after liposome injection. Analysis of SN-38 levels in tumor tissues was as described by Noble, et al, Cancer Res. 2006;66:2801-2806. Results (FIG. 3) show no differences in tumor SN-38 levels between FMX untreated controls and FMX pre-treated animals, demonstrating that FMX has no effect on the pharmacodynamics of liposomal irinotecan, particularly MM-398.

### EXAMPLE 4

### Effects of Liposomal Iodine on Pharmacology of Subsequently Administered Antibody Targeted Liposomal Doxorubicin

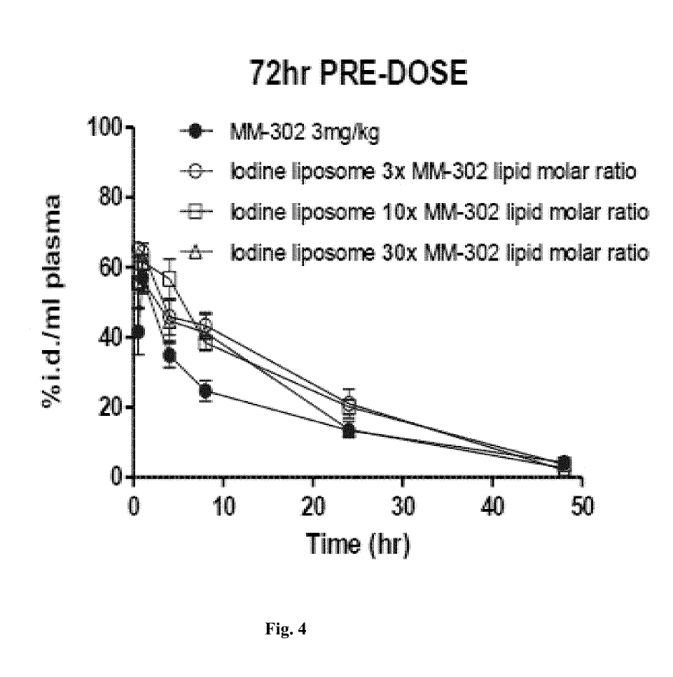

HT-29 tumor-bearing mice were injected with imaging agent followed by injection of liposomes as described in the preceding Example, except that the imaging agent was nanoliposomal iodine and the therapeutic liposomes were MM-302 liposomal doxorubicin at a dosage of 3 mg/kg. The liposomal iodine was prepared by encapsulating iodixanol (LGM Pharma) in cholesterol, HSPC, PEG-DSPE (55:40:5) liposomes with an average diameter of from 110 to 170 nm and a final iodine concentration of 6.3×10⁵ to 2.1×10⁶ iodine molecules per liposome and was administered at levels indicated in FIG. 4. Results obtained indicate that pretreatment of mice with an iodine-loaded liposomal contrast agent dramatically reduced the clearance of a subsequent dose of HER2-targeted liposomal doxorubicin (MM-302) even three days after administration of a liposomal iodine contrast agent (FIG. 4). The 30× concentration is the concentration of liposomal iodine contrast agent required to give a low level of contrast for CT imaging.

### EXAMPLE 5

### FMX Measurement by MRI and Drug Deposition in Different Tumor Models

In order to correlate MRI-based measurements of FMX deposition with irinotecan drug levels in tumor tissues after deposition of liposomes, three human tumor cell lines (HT29, A2780 and A549) were used to establish subcutaneous flank tumor xenografts growing bilaterally in SCID mice (n=4 for each model). Tumor-bearing mice thus prepared were injected intravenously with FMX at 20 mg/kg.

Mice were imaged at baseline with a T29/T2*MRI just before the FMX injection and again at 24 h and 72 h after the injection. The average T2 rate was determined for each tumor volume. The FMX concentration at each time point was determined from the difference relative to the baseline of the relaxation rates divided by the known relaxivity parameter r2. Mice were then administered a single dose of MM-398 equivalent to 10 mg irinotecan HCL/kg. Tumor samples were taken after 24 h or 72 hr of that injection and analyzed for irinotecan levels by HPLC as described above in Example 3.

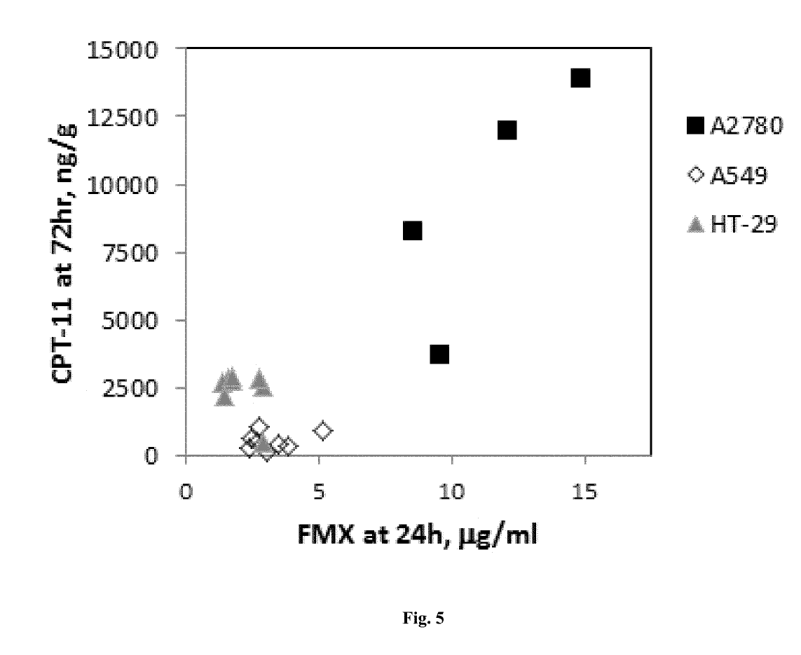

Results (FIG. 5) show that especially for individual A2780-derived tumors the FMX concentration measured at 24 hr after FMX injection correlated well with the irinotecan (CPT-11) drug levels measured at 72 h in the lysates of the same tumors. The FMX concentrations in the A549 and HT29-derived tumors established a lower signal threshold for FMX-based MRI that correlated with lower irinotecan concentrations.

### EXAMPLE 6

### Non-Invasive Imaging with Ultrasmall Superparamagnetic Iron Oxide Particles (USIOPs) in Patients

### Materials and Methods

### Administration of FMX

As disclosed herein, FMX can robustly and accurately predict delivery, and thus activity, of nanotherapeutics that have a t1/2 of clearance of greater than 6 hours, greater than 10 hours, and greater than 18 hours in mice (or 12, 24, and 48 hours in humans).

The nanotherapeutic may be, e.g., a liposome-encapsulated small molecule drug or prodrug, e.g., a ligand-targeted liposome-encapsulated drug. The prodrug may be an ester, e.g., an ester that requires conversion by one or more esterases for activation. The encapsulated drug or prodrug may be, e.g., a taxane, a tyrosine kinase inhibitor (TKI), or a camptothecin. An exemplary camptothecin prodrug is irinotecan.

### USIOPs

USIOPs having particles ranging from 10-50 nm in size and exhibiting a half-life of clearance from the bloodstream of at least 10 hours are suitable for use in the imaging techniques disclosed herein. An exemplary USIOP for use in the disclosed methods is ferumoxytol (FMX).

### Dosage

A minimum dosage in humans of USIOPs for the imaging methods disclosed herein comprises 3 mg/kg of iron. In some embodiments, the dose is at least 5 mg/kg. In other embodiments, the dose is at least 10, at least 15, or up to 20 mg/kg of iron. In one embodiment, the patient is an adult, the USIOP is FMX, and the dose is a 510 mg dose.

### Prescreening Prior to FMX Treatment

A patient's iron levels are measured in the blood prior to ferumoxytol administration. Iron overload is diagnosed by measuring a fasting morning transferrin saturation ≧45% (ratio of serum iron divided by the serum total iron binding capacity and expressed as a percentage). A ferritin level of 1000 ng/ml is likely to be also associated with organ damaging levels of iron. Blood is collected from each patient and the plasma is analyzed for transferrin according to standard Clinical Laboratory protocols. Administration of USIOPs is contraindicated in patients with iron overload and such patients should not be imaged in accordance with the methods disclosed herein. For all patients, ferumoxytol is used in accordance with the manufacturer's label warnings.

### Dosage and Administration of FMX

Ferumoxytol (30 mg/mL) is available for intravenous injection in single use vials. Each vial contains 510 mg of elemental iron in 17 mL.

A single dose of ferumoxytol will be administered at Day 1 by intravenous injection. Dosing is calculated according to patient weight at 5 mg/kg. The total single dose does not exceed 510 mg, the maximum approved single dose of ferumoxytol. Ferumoxytol is administered as an undiluted IV injection at a rate of up to 1 ml (30 mg) per second with monitoring of vital signs.

### Magnetic Resonance Imaging (MRI)

The MRI image is obtained using a magnetic field strength of 1-10 Tesla. For example, a magnetic field strength of 1.5 or 3 Tesla is used. Basic MRI scan sequences for the acquisition of T1-weighted, T2-weighted and T2*-weighted images are used to measure FMX-dependent T1 hyper-enhancement or T2/T2*-signal hypoenhancement (contrast enhancement due to the excellent relaxivity properties of this USPIO). The r₂ relaxivity for FMX at 1.5 T, 37° C. in water or plasma is reported to be r₂=89 mM⁻¹ sec⁻¹. The relaxivity parameter can be used to quantify the signal contrast in FMX images by either T2(^{*}) or R2(^{*}) (R2=1/T2) analysis methods. The relationship between the R₂(^{*}) relaxation rates, the r₂(^{*}) relaxivity parameter and the FMX concentration is given by the following equation [1].

$${R}_{{2}^{*}} = \frac{1}{{T}_{{2}^{*}}} = {( {R}_{{2}^{*}} )}_{0} + ( {r}_{{2}^{*}} ) \cdot [ FMX ] \qquad [ 1 ]$$

Imaging timepoints

For prediction of liposomal deposition and activity in humans, imaging is typically performed initially pre-FMX administration to establish a background level of contrast and then subsequently between 1 and 168 hours (0-7 days) following FMX administration. For prediction of activity by nanocarriers that are dependent on macrophage uptake (e.g., non-targeted and/or prodrug-containing nanocarriers), the imaging will be performed between 1-7 days post-FMX dosing. Finally, for targeted nanotherapeutics that do not rely primarily on macrophage uptake for uptake, processing and/or conversion, the imaging may beneficially be carried out earlier, e.g., between 20 and 48, or 24 and 72 hours post-FMX dosing.

### Imaging Procedure

In an exemplary embodiment, FMX is administered to a patient being considered for nanoliposomal therapy for a tumor. First a pre-dose MRI (MRI1) is performed to established baseline. Following FMX administration one or more additional MRIs is performed.

A second MRI (MRI2) is (optionally) performed (1.5-4 hours after FMX administration) to record tumor blood volume.

A third MRI (MRI3) is (optionally) performed approximately 24 hours after FMX administration. Liposome tumor deposition peaks around 24 hours after administration and FMX signal should be also predominately tumor-based (as FMX should have been at least partially cleared from the blood). Therefore, MRI3 results (corrected for MRI1 and MRI2) are correlated with liposome deposition potential.

A fourth MRI (MRI4) is (optionally) performed approximately 72 hours after FMX administration. FMX accumulation in macrophages follows deposition, and is expected to peak between 24 and 72 hours; therefore, MRI4 signal enhancement should correlate with USIOP uptake by TAMs.

The difference in contrast between MRI3 and MRI1 is calculated to provide an estimate of the amount of deposition that occurred and therefore what can be expected for a liposome therapy. If deposition is undetectable, or is lower than a preset threshold (e.g., at least 5%, 10%. 15%, 20%, or 25% over MRI1) a patient should be excluded from nanoliposomal therapy.

The difference in contrast between MRI2, MRI3, or MRI4 and MRI1 is calculated to provide an estimate of the USIOP uptake by TAMs, which is believed to correlate with the liposome drug release potential for a liposome therapy at the location of USIOP uptake. If the liposome drug release potential is lower than a preset threshold (e.g., at least 5%, 10%. 15%, 20%, or 25% over MRI1) a patient is excluded from receiving nanoliposomal therapy.

As shown with equation [1] above, the difference in contrast can be related to FMX concentrations at a specific site or sample location.

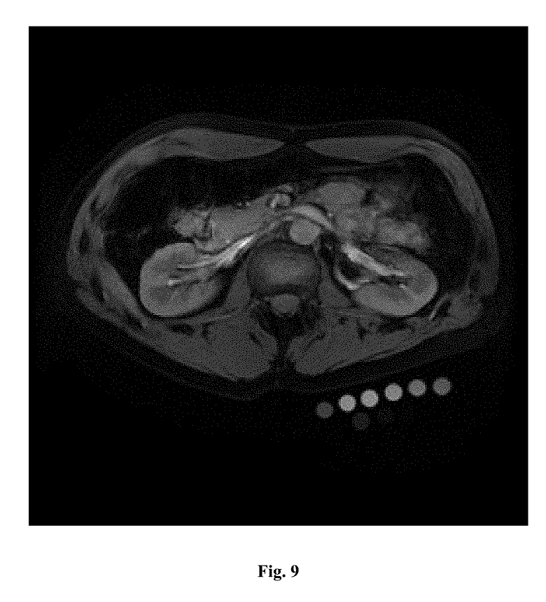

Standards (“phantoms”) of different concentrations of FMX ranging from 0-200 μg/ml with the FMX being suspended in 2% agarose or similar gels may be included within the MR imaging cavity (e.g., in tubes or vials), so that image acquisition of the standards can occur while imaging a patient. These imaging data can provide reference points for the quantitation of FMX. An exemplary image of such “phantoms” is shown in FIG. 9, which shows a T2* weighted MRI with TE of 13.2 ms with phantom tubes from 0-200 μg/ml ferumoxytol.

### EXAMPLE 7

### MRI Imaging of FMX in Patient Plasma and Tumor Lesions and use of MRI-Derived FMX Data in a Pharmacokinetic Model

In a human clinical trial (ClinicalTrials.gov Identifier: NCT01770353) patients with advanced solid tumors and multiple metastases were injected with FMX at 5 mg/kg and imaged in an MRI scanner as described in Example 6. Blood was drawn before and at about 30 min, 2 hr, 24 hr and 72 hr after the injection of FMX. Plasma was prepared according to published protocols. Plasma FMX concentrations for each patient were determined from MRI images of plasma imaged together with an FMX “phantom” standard. Analysis of T2*-weighted images in comparison to an external “phantom” standard as described in Example 6 allowed the extrapolation of FMX concentrations in tumor lesions. Plasma and tissue FMX concentrations from individual lesions were input into a pharmacokinetic model of both compartments (MATLAB) that considers a mixed model of FMX deposition and allows estimation of tissue-specific parameters of FMX-dependent tissue permeability and signal retention/tissue binding. These two parameters can thus characterize individual lesions.

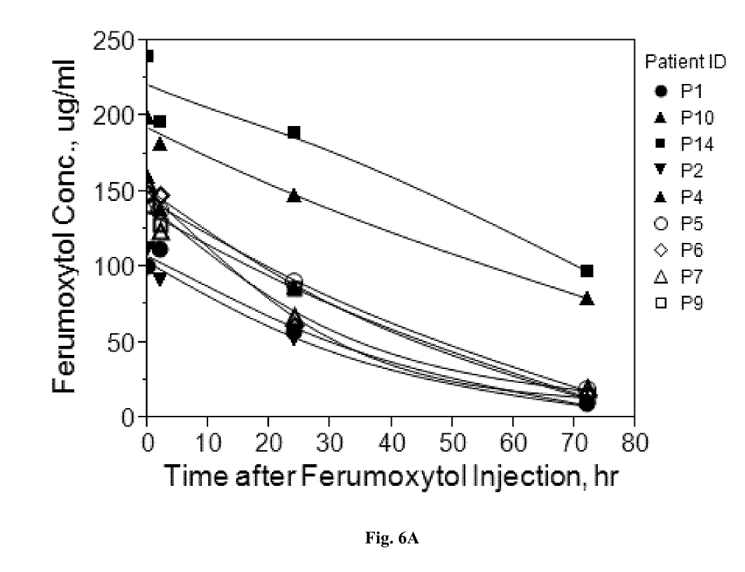

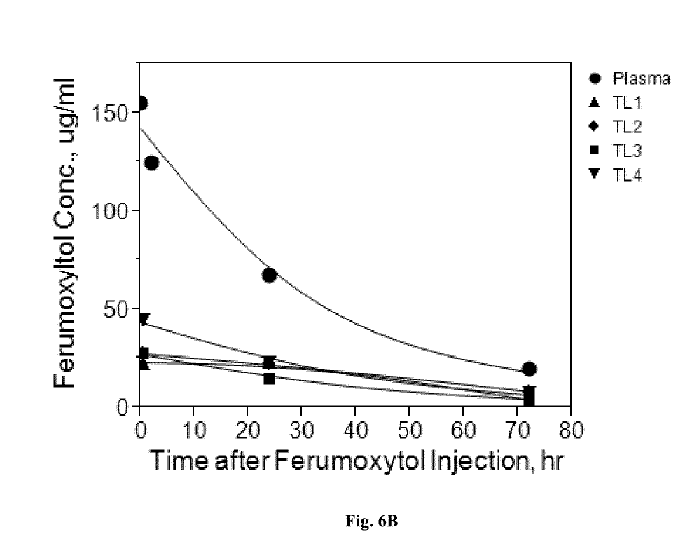

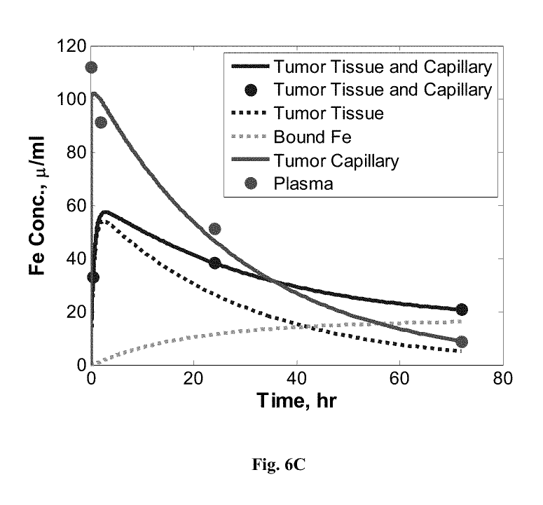

Results (FIG. 6A) show that plasma levels of FMX decline consistent with published data in healthy volunteers (Landry et al., Am. J. Nephrol. 2005; 25(4):400-10). In some patients FMX clearance was low resulting in higher FMX levels in both plasma and tumor lesions. As shown in FIG. 6B the FMX signal in lesions from a representative patient show an immediate perfusion-dependent uptake of FMX, but levels are maintained or even increase at the 24 h MRI measurement. FMX clearance was slower in tumor lesions than in the plasma. A pharmacokinetic model (see FIG. 6C) identifies lesion-characteristic tissue permeability and tissue binding parameters that together allow a fit to the measured FMX levels in individual lesions. The permeability parameter contributes to early FMX signals, while the binding parameter contributes to later FMX signals.

### EXAMPLE 8

### Deposition of FMX in Patient Tumor Lesions and Co-Localization with Macrophages

Tumor biopsies from the NCT01770353 clinical trial outlined in Example 7 were obtained 72 h and 168 hrs after FMX injection at 5 mg/kg and immediately fixed in formalin. Serial tumor sections from formalin fixed paraffin embedded (FFPE) tissue were prepared and assessed by Prussian Blue staining for FMX and by staining with anti-CD68 antibody (clone PG-Ml, DAKO) for macrophages. Sections were imaged with an Aperio® ScanScope AT® whole slide scanner.

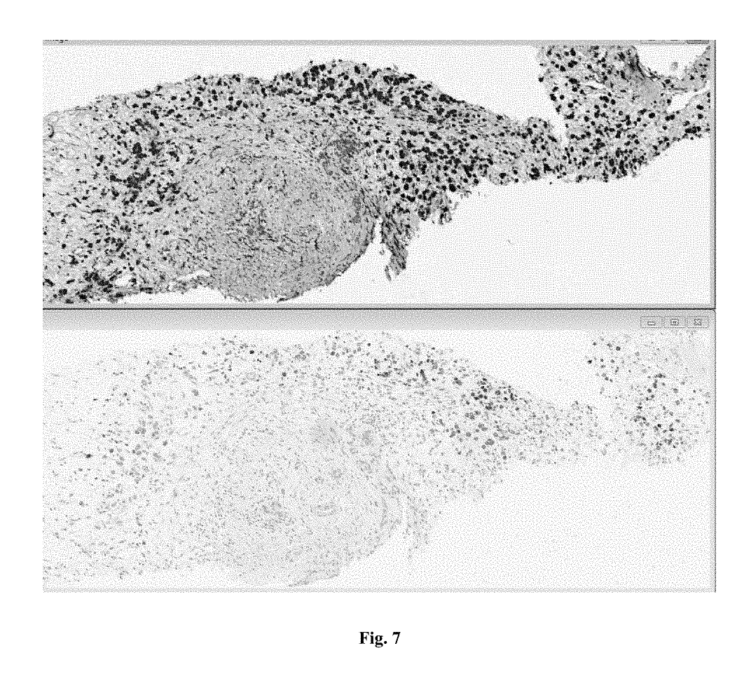

Results (FIG. 7) show that FMX deposition was detectable primarily in stromal areas around tumor nests. The staining pattern suggests intracellular FMX accumulation that is highly co-localized with macrophages stained in adjacent sections. This association was observed in biopsies obtained at 72 h and 168 h and suggests that FMX deposition can identify vascular-accessible macrophages within tumor lesions.

### EXAMPLE 9

### Drug Levels in Biopsies Targeted Towards Lesion Areas with Different Levels of FMX Signal Hypoenhancement

In the NCT01770353 clinical trial described above, patients with advanced solid tumors and multiple metastases were injected with FMX at 5mg/kg and imaged in an MRI scanner. The T2*-weighted images from all time-points were used to outline lesion-specific regions of interest (ROI) with higher or lower levels of FMX-dependent signal hypoenhancement. MRI image analysis extrapolated FMX levels from these ROI within the lesions. CT-guided needle core biopsies were directed towards the same ROI, and the localization of the percutaneous sampling site was projected back onto the FMX MRI images by multimodality image fusion for optimization of the ROI outline. This percutaneous sampling was done using manual control. Tissue collection was performed 72 hr after the infusion of 80 mg/m² MM-398, which coincided with 168 hr after the injection of FMX. Core biopsies were frozen. For measurements of the irinotecan metabolites SN-38 and SN-38G, samples were weighted and homogenized in a 50% methanol/water mixture in a bullet blender. After extraction with acidified methanol, the supernatant was collected, dried down, reconstituted in mobile phase solution and analyzed on an LC/MS/MS TSQ Vantage (Thermo Scientific) system.

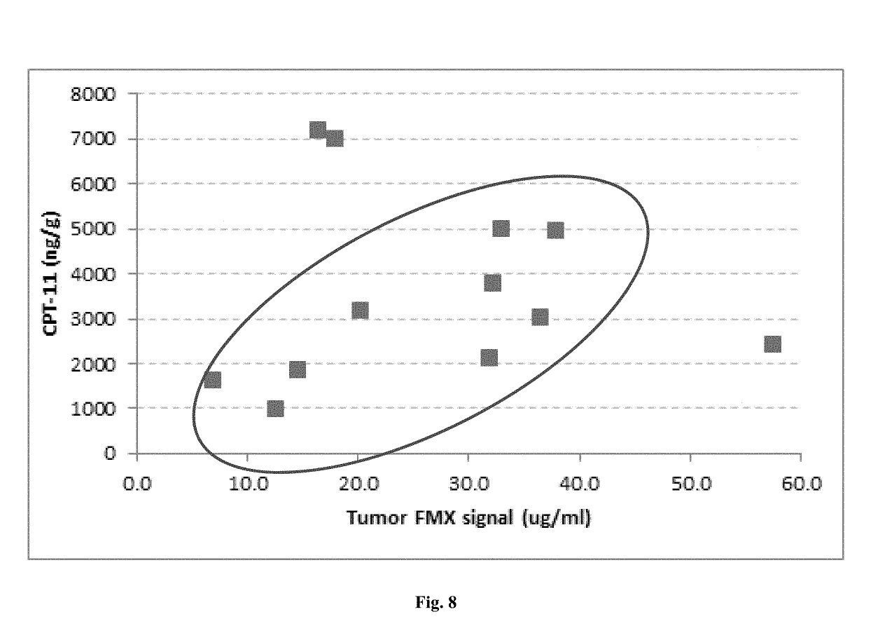

Results (FIG. 8) show the tumor FMX signal measured at 24 hr in ROI with higher or lower levels of FMX-dependent signal hypoenhancement (prior to ROI outline optimization) and the irinotecan levels measured in biopsies from the vicinity of the same ROI. For a majority of biopsy samples there is a correlation between the level of the 24 h FMX signal and the irinotecan level.

### EXAMPLE 10

### MRI Imaging of FMX in Patient Lesions and Comparison to Treatment-Dependent Changes in Lesion Volume

In the NCT01770353 clinical trial described above, patients with advanced solid tumors and multiple metastases were injected with FMX at 5 mg/kg and imaged in an MRI scanner as described in Example 6. Patient lesions were also imaged by X-ray Computed Tomography at the beginning of treatment (“Screening”) and after four weeks (“EOC2”). The individual lesion volumes were calculated at both time points.

Results summarized in the table below show the measured FMX levels at day 2 (24 h) and day 4 (72 h) after FMX injection together with the calculated volumes of four liver lesions at treatment start (“Screening”) and after four weeks or two treatment cycles (“EOC2”). All four lesions showed a decrease in measurable lesion size. Compared to the median FMX levels in 26 lesions of seven patients the 24 h FMX levels are all elevated, but the levels in lesion 2 are lower than in the other three lesions. The 72 h FMX levels for lesions 2-4 are above the median FMX levels, but have dropped below the median in lesion 1. Lesion 1 was the smallest lesion at treatment start, lesion 2 was the largest lesion at treatment start.

| | Day 2 | Day 4 | | | |
|---|---|---|---|---|---|
| | Fe-MRI | Fe-MRI | Screening CT | EOC2 | Vol |
| Lesion | (μg/ml) | (μg/ml) | (cc) | (cc) | change |
| 1 | 38.98 | 6.11 | 3.7 | 1.0 | −73% |
| 2 | 28.04 | 12.8 | 44.3 | 30.3 | −32% |
| 3 | 39.94 | 14.37 | 9.2 | 1.6 | −83% |
| 4 | 37.35 | 13.09 | 10.3 | 2.0 | −81% |
| Median all | 22.93 | 10.84 | | | |
| lesions* | | | | | |

*Patients 0001-0009 (26 lesions)

### INCORPORATION BY REFERENCE

Each and every issued patent, patent application and publication specifically referred to herein is hereby incorporated herein by reference in its entirety.

1. A method for selecting and providing pharmaceutical treatment to a patient for a localized infectious, inflammatory, or neoplastic condition, the method comprising:
diagnosing a localized infectious, inflammatory, or neoplastic condition in a patient and
identifying one or more locations of infection, inflammation or neoplasia in the patient, and subsequently,
obtaining a first contrast-enhanced MRI image of a first location of the one or more locations, and subsequently,
selecting an anti-infective, anti-inflammatory, or anti-neoplastic pharmaceutical agent and treating the patient with the selected pharmaceutical agent;
wherein,
the first contrast-enhanced MRI image is obtained following administration of an MRI contrast agent comprising an MRI contrast-enhancing amount of superparamagnetic iron oxide nanoparticles of about 15-30 nm in diameter, and
if the first contrast-enhanced MRI image shows contrast enhancement within the first location, the selected pharmaceutical agent is an agent that is formulated as nanoparticles or nanoliposomes of about 70 to about 200 nm in diameter, but
if the first contrast-enhanced MRI image does not show contrast enhancement at first location, the selected pharmaceutical agent is an agent that is not formulated as nanoparticles or nanoliposomes of about 70 to about 200 nm in diameter.

2. The method of claim 1, wherein the contrast agent is ferumoxytol.

3. The method of claim 2, wherein the ferumoxytol is administered at 5 mg/kg.

4. The method of claim 2, wherein the patient is an adult and 100 1000 milligrams of ferumoxytol are administered to the patient.

5. The method of claim 2, wherein the patient is an adult and 510 milligrams of ferumoxytol are administered to the patient.

6. The method of claim 1, wherein the contrast-enhanced MRI image is obtained no more than 24, 48, 72, 96, 120, 144 or 168 hours after administration of the superparamagnetic iron oxide nanoparticles.

7. The method of claim 1, wherein the contrast-enhanced MRI image is obtained from 24-48, 48-72, 72-96, 96-120, 120-144, or 144 to 168 hours after administration of the superparamagnetic iron oxide nanoparticles.

8. A method for selecting and providing pharmaceutical treatment to a patient for a localized infectious, inflammatory, or neoplastic condition, the method comprising:
diagnosing a localized infectious, inflammatory, or neoplastic condition in a patient and
identifying one or more locations of infection, inflammation or neoplasia in the patient, and subsequently,
obtaining a first contrast-enhanced MRI image of a first location of the one or more locations,
obtaining a second contrast-enhanced MRI image of a second location of the one or more locations, and subsequently,
selecting an anti-infective, anti-inflammatory, or anti-neoplastic pharmaceutical agent and treating the patient with the selected pharmaceutical agent;
wherein,
the first and second contrast-enhanced MRI images are obtained following administration of an MRI contrast agent comprising an MRI contrast-enhancing amount of superparamagnetic iron oxide nanoparticles of about 15-30 nm in diameter, and
if both the first and the second contrast-enhanced MRI images show contrast enhancement within both the first location and second location, the selected pharmaceutical agent is an agent that is formulated as nanoparticles or nanoliposomes of about 70 to about 200 nm in diameter, but if both the first and the second contrast-enhanced MRI images do not show contrast enhancement within both the first location and second location, the selected pharmaceutical agent is an agent that is not formulated as nanoparticles or nanoliposomes of about 70 to about 200 nm in diameter.

9. The method of claim 8, wherein the contrast agent is ferumoxytol and 1) is administered at 5 mg/kg, or 2) is administered to an adult patient at a dosage of 100-1000 milligrams, or 3) is administered to an adult patient at a dosage of 510 milligrams.

10. The method of claim 1, wherein the MRI image is obtained using a magnetic field strength of 1-10 Tesla or about 1.5 Tesla, or about 3 Tesla.

11. The method of claim 1, wherein the liposomal therapeutic agent comprises liposomes of an average size of about 100 nm in diameter with an average range of about 75 to about 200 nm or of about 80 to about 120 nm.

12. (canceled)

13. The method of claim 1, wherein the liposomal therapeutic agent is MM-302.

14. The method of claim 1, wherein the first contrast-enhanced MRI image shows contrast enhancement within the first location, the neoplastic condition comprises at least one tumor and the pharmaceutical agent is a liposomal anti-neoplastic agent.

15. The method of claim 14 wherein the tumor is a non-small cell lung cancer (NSCLC) tumor, a triple negative breast cancer (TNBC) tumor, a colorectal cancer (CRC) tumor, a pancreatic cancer tumor, a small cell lung cancer tumor, a gastric cancer tumor, a cervical cancer tumor, or Ewing's sarcoma and the selected pharmaceutical agent is a liposomal formulation of irinotecan.

16. (canceled)

17. The method of claim 14 wherein the liposomal anti-neoplastic agent is liposomal doxorubicin.

18. The method of claim 17 wherein the liposomal doxorubicin comprises antibody-targeted doxorubicin liposomes.

19. The method of claim 18 wherein the antibody-targeted doxorubicin liposomes are targeted with an anti-HER2 antibody.
